(19) United States
(12) Patent Application Publication
(10) Pub. No.: US 2023/0343443 A1
(43) Pub. Date: Oct. 26, 2023

(54) EMERGENCY MEDICAL SYSTEM FOR HANDS-FREE MEDICAL-DATA EXTRACTION, HAZARD DETECTION, AND DIGITAL BIOMETRIC PATIENT IDENTIFICATION

(71) Applicant: MOTOROLA SOLUTIONS, INC., Chicago, IL (US)

(72) Inventors: DAVID WEINRICH, TEMECULA, CA (US); ILYA ORLIK, ENGLEWOOD, CO (US); JOSEPH NAMM, PLANTATION, FL (US); CHRISTOPHER E BLACKSHEAR, HOFFMAN ESTATES, IL (US); CHONG KEAT CHUA, Ayer Itam (MY)

(21) Appl. No.: 17/659,887

(22) Filed: Apr. 20, 2022

(51) Int. Cl. G16H 40/20; G16H 40/67; G16H 10/60; G06V 40/16; G10L 17/00; G06F 40/40; H04W 4/90

(52) U.S. Cl. CPC G16H 40/20; G16H 40/67; G16H 10/60; G06V 40/16; G10L 17/00; G06F 40/40; H04W 4/90

(57) ABSTRACT

Systems described herein can use microphones and cameras to capture media data (e.g., audio data and video data) at the scene of an emergency, apply specific types of software to the media data to extract medical data for a patient and a digital biometric identifier of the patient, associate the medical data with the digital biometric identifier in a data structure, and transmit the medical data, the digital biometric identifier, and the data structure to a treatment facility. The systems described herein can accomplish these tasks without involving responders' hands, thereby allowing responders' hands to be used for treating patients, driving ambulances, and performing other lifesaving tasks without interruption. When a person arrives at the treatment facility, a second digital biometric identifier can be captured and compared to the digital biometric identifier to confirm that the person is the patient to whom the medical data applies.

### BACKGROUND

Emergency calls (e.g., 9-1-1 calls) may be routed to specialized call centers known as public safety answering points (PSAPs). Dispatchers at the PSAPs answer the emergency calls, assess the nature of the emergencies being reported by those calls, and dispatch appropriate emergency-response personnel accordingly. In particular, when a medical emergency arises, an ambulance may be dispatched to the scene of the emergency to treat people who have been seriously injured, have suffered a medical episode (e.g., a heart attack, a stroke, or a seizure), or have been victims of other types of life-threatening events. Upon arriving at the scene, paramedics may assess the conditions of victims who need assistance and provide medical treatment to the extent possible at the scene and in the ambulance while transporting victims to treatment centers (e.g., hospitals) where doctors are equipped with specialized diagnostic equipment (e.g., x-ray machines, Magnetic Resonance Imaging (MRI) machines, chemistry analyzers, immunoassay analyzers, assay kits, and hematology analyzers) and are able provide lifesaving treatment (e.g., surgery) that paramedics are not equipped to provide at the scene.

### BRIEF DESCRIPTION OF THE SEVERAL VIEWS OF THE DRAWINGS

In the accompanying figures similar or the same reference numerals may be repeated to indicate corresponding or analogous elements. These figures, together with the detailed description, below are incorporated in and form part of the specification and serve to further illustrate various examples of concepts that include the claimed invention, and to explain various principles and advantages of those examples.

FIG. 1 illustrates an example computing environment in which systems of the present disclosure can operate, according to one example.

FIG. 2 illustrates functionality for systems disclosed herein, according to one example.

FIG. 3 illustrates additional functionality for systems disclosed herein, according to one example.

FIG. 4 illustrates a schematic block diagram of a computing device, according to one example.

Skilled artisans will appreciate that elements in the figures are illustrated for simplicity and clarity and have not necessarily been drawn to scale. For example, the dimensions of some of the elements in the figures may be exaggerated relative to other elements to help improve understanding of examples of the present disclosure.

The system, apparatus, and method components have been represented where appropriate by conventional symbols in the drawings, showing only those specific details that are pertinent to understanding the examples of the present disclosure so as not to obscure the disclosure with details that will be readily apparent to those of ordinary skill in the art having the benefit of the description herein.

### DETAILED DESCRIPTION OF THE INVENTION

When medical emergencies occur, time is of the essence. For this reason, most jurisdictions have laws that preempt general automotive traffic rules when the sirens and flashing lights of ambulances are activated (e.g., ambulances are allowed to exceed speed limits, drive on freeway shoulders, and run red lights) so that ambulances can arrive at accident scenes quickly. Furthermore, most jurisdictions mandate that civilian drivers move vehicles aside to clear a path for ambulances that are en route to the scenes of emergencies or are transporting patients to treatment centers (e.g., hospitals). In addition to laws, many different technologies are also used to facilitate fast ambulance responses. In Enhanced 911 (E911) systems, for example, when 9-1-1 is dialed from a cellular phone, the wireless network through which the cellular phone operates provides the location of the cellular phone (e.g., as determined by a Global Positioning System (GPS) receiver in the cellular phone or by radiolocation techniques such as triangulation between cellular towers) to the PSAP that receives the call so that an ambulance can be apprised of the caller's location quickly even if the caller is unable to specify the location.

Once an ambulance arrives at the scene of an emergency, the urgency of the situation may oblige responders such as paramedics and Emergency Medical Technicians (EMTs) to prioritize tasks related to the emergency response. For example, if a patient is bleeding profusely when an ambulance arrives at the scene, one responder may be obliged to apply pressure (e.g., to an artery) to slow the bleeding while another responder may be obliged to retrieve a gurney from the ambulance, lower the gurney, load the patient onto the gurney, raise the gurney, and wheel the patient into the ambulance while the other responder continues applying pressure. One responder may continue applying pressure to mitigate the bleeding while another responder drives the ambulance to a hospital. In another example, one responder may be obliged to apply a non-rebreather mask to a patient's mouth and nose while another responder attaches the electrodes of an automated external defibrillator (AED) to the patient's body. In another example, one responder may be obliged to apply a cervical collar (i.e., a neck brace) to a patient's neck while another responder secures a traction splint to one of the patient's limbs. Legions of other scenarios exist in which situational exigencies may give responders' hands little opportunity to be engaged in less-urgent tasks.

Frequently, while the responders are treating patients, responders are also concurrently gathering information about the patient's condition and identity. In some scenarios, a patient may be conscious—at least initially—and able to respond verbally to questions posed by the responders. For example, the patient may be able to state the patient's name, the cause of the patient's injuries (e.g., automobile accident, explosion, gunshot wound, stabbing, etc.), the symptoms the patient is experiencing (e.g., headaches, pain, numbness in limbs, hallucinations, etc.), and additional information that may be pertinent to how the patient should be treated (e.g., whether the patient has a bleeding disorder such as hemophilia, whether the patient is on a blood thinner such as Warfarin, whether the patient is allergic to antibiotics or other medicines, whether the patient is diabetic, whether the patient is pregnant, whether the patient is currently taking any medications, whether the patient is under the influence of alcohol or any other drug, and whether the patient suffers from medical conditions such as asthma, osteogenesis imperfecta, or an autoimmune disorder). Patients who are injured severely enough to be transported to a hospital, though, may already be unconscious when an ambulance arrives or may be in danger of losing consciousness at any moment.

Regardless of whether a patient is conscious, responders may also be tasked with communicating urgent information to each other and to a dispatcher verbally throughout the process of treating and transporting the patient. For example, one responder may confirm to another that a patient's systolic blood pressure is greater than 90 millimeters of mercury (mm HG) so that another responder will know that nitroglycerin may be administered for the patient's chest pain if the patient confirms that no phosphodiesterase inhibitors have recently been taken by the patient. Responders may further state when drugs such as epinephrine (e.g., for anaphylaxis), albuterol (e.g., for asthma), Naloxone (i.e., Narcan) (e.g., for opioid overdoses), and tranexamic acid (TXA) (e.g., for major hemorrhages) are being administered to a patient and how much of each drug is being administered. Responders may also apprise a dispatcher of hazardous conditions that exist at the scene so that the dispatcher can advise other responders who are still en route to the scene. For example, if responders at the scene tell a dispatcher that some of the patients at the scene are under the influence of a transdermal drug such as phencyclidine (PCP) or Fentanyl, the dispatcher may advise responders that are en route not make contact with the patients' skin. In another example, if the responders at the scene tell the dispatcher that a structural fire is spreading toward a gas meter or a tank of a flammable substance, the dispatcher may advise responders who are in route of the potential for an explosion.

In addition, conveying the information that responders gather to the treatment facilities that will receive patients who are transported by ambulance may be a matter of life and death. Therefore, responders are generally responsible for documenting medical data (e.g., the nature of injuries, any drugs taken by or administered to the patient, etc.) for patients and the identities of patients and providing that documentation to the treatment centers that receive the patients. However, as explained above, the hands of first responders are often compelled to engage in urgent, lifesaving treatment. If a first responder is obliged to stop administering treatment to a patient in order to record medical data (e.g., by typing on a keyboard or writing by hand on a clipboard), valuable time may be consumed by the both the recording process and by other factors (e.g., if responders are obliged to wash blood from their hands before handling a keyboard or clipboard). When seconds count—as is frequently the case in medical emergencies to which ambulances respond—delays that might seem innocuous in other contexts may pose a serious danger for patients. It might be possible for responders to communicate medical data and identity data for patients to hospital staff verbally when an ambulance delivers a patient, but this may result in a delay in the ambulance's response time for another imminent medical emergency.

When responding to events such as mass shootings, structural collapses (e.g., buildings or bridges), structural fires, explosions (e.g., terrorist bombings, gas line explosions, and industrial explosions), multi-car pileups, natural disasters (e.g., tornadoes, hurricanes, earthquakes, avalanches, landslides, and tsunamis), and other mass casualty incidents (MCIs), the challenges first responders face may be multiplied and amplified. A single team of responders may be obliged to provide treatment to multiple patients according to a triage protocol. As many patients may be suffering from serious injuries, responders may be obliged to postpone documentation tasks in favor of tending to the imminent physical needs of patients. The scene of the emergency may be chaotic and many patients may be unconscious. It may be difficult to determine the identities of each patient and to keep the medical data for each patient properly associated with the patient's identity. After a mudslide, for example, it may be impractical to identify patients based on articles of clothing because many patients' clothing and faces may be covered with mud. In another example, if a bomb is detonated in a hotel where an annual conference was being held, many patients may be dressed very similarly (e.g., in tailored suits). Patients at the scene of a structural fire may also be difficult to distinguish from one another as clothing and faces may be obscured by soot. In such circumstances, if one patient's medical data is mistakenly associated with another patient, both patients may suffer life-threatening consequences.

For example, suppose a patient at the scene of a fire in a multistory condominium informs a responder that the patient has a leadless pacemaker (which was implanted via a catheter and therefore left no scar on the patient's chest) before the patient passes out. Also suppose the responder is obliged to assist several other patients shortly after the patient with the pacemaker loses consciousness. If, in the confusion, the responder loses track of which soot-covered patient has the pacemaker, the patient with the pacemaker may be endangered if an AED is applied or if hospital personnel put the patient into a magnetic resonance imaging (MRI) machine without knowing that the patient has the pacemaker (e.g., because the pacemaker contains metal). Furthermore, an additional patient mistaken for the patent with the pacemaker may be endangered if responders refrain from applying an AED to the additional patient due to the mistaken belief that the additional patient has a pacemaker.

Thus, despite the many existing technologies that are used to help first responders perform lifesaving tasks quickly, there are still many challenges that can cause potentially lethal delays for responders—challenges that existing technologies are inadequate to address. For example, as mentioned above, the exigency of gathering and documenting potentially lifesaving information may directly compete with the exigency of providing lifesaving treatment to patients for responders' time and attention. As patient survival may hinge on having responders successfully address both of these exigencies, responders may be faced with the dilemma of whether to neglect one exigency in order to address the other. Furthermore, in some scenarios (e.g., MCIs), even if accurate medical data is collected, it may prove challenging to associate that medical data with the proper patients unambiguously and in a form that can be transferred readily to treatment facilities within the tight time constraints to which responders are often subjected.

Systems, methods, and devices of the present disclosure provide solutions that leverage multiple technologies to address these challenges. For example, systems described herein can use microphones and cameras to capture media data (e.g., audio data and video data) at the scene of an emergency, apply specific types of software to the media data to extract medical data for a patient and a digital biometric identifier of the patient, associate the medical data with the digital biometric identifier in a data structure, and transmit the medical data, the digital biometric identifier, and the data structure to a treatment facility. The systems described herein can accomplish these tasks without involving responders' hands, thereby allowing responders' hands to be used for treating patients, driving ambulances, and performing other lifesaving tasks without interruption.

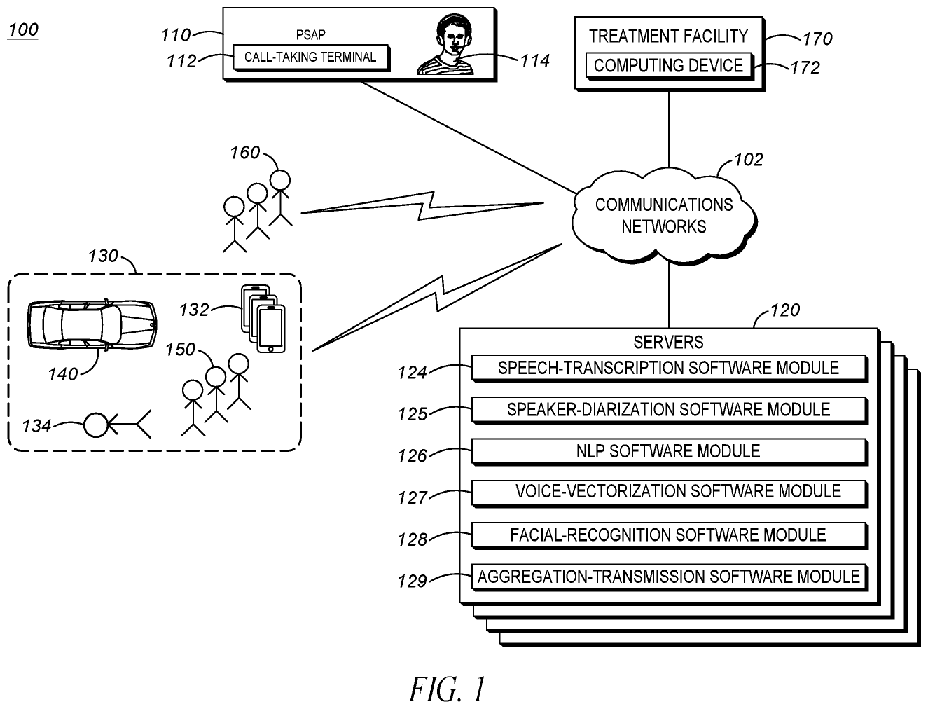

FIG. 1 illustrates an example computing environment 100 in which systems of the present disclosure can operate, according to one example. As shown, the computing environment 100 includes communications networks 102 that allow electronic data to be exchanged between a call-taking terminal 112 at a PSAP 110, a computing device 172 in a treatment facility 170, mobile devices 132, and servers 120 via wireless connections, wired connections, or a combination thereof. The communications networks 102 may comprise, for example, a wide area network, the Internet (including public and private Internet Protocol (IP) networks), a Long Term Evolution (LTE) network, a Global System for Mobile Communications (or Groupe Special Mobile (GSM)) network, a Code Division Multiple Access (CDMA) network, an Evolution-Data Optimized (EV-DO) network, an Enhanced Data Rates for Global Evolution (EDGE) network, a Third Generation Partnership Project (3GPP) network, a 4G network, a 5G network, a landline telephonic network, a Low Earth Orbit (LEO) network (e.g., for satellite phones), a Geosynchronous Orbit (GEO) network (e.g., for satellite phones), a local area networks (LANs) (e.g., a Bluetooth™ network or a Wi-Fi network), an enterprise network, a data-center network, a virtual private network (VPN), or combinations thereof. The communications networks 102 may further facilitate the use of applications that implement mobile alliance (OMA) push to talk (PTT) over cellular (OMA-PoC), VoIP, or PTT over IP (PoIP).

To illustrate how the systems of the present disclosure may operate in the computing environment 100, consider the following example. Suppose an emergency call received at the PSAP 110 (e.g., via the communications networks 102) alerts the dispatcher 114 (who is working at the call-taking terminal 112) of an emergency situation that is unfolding at a scene 130. In response, the dispatcher 114 uses the call-taking terminal 112 to dispatch an ambulance 140 to scene 130. Responders 150 (e.g., paramedics or EMTs) arrive at the scene 130 and discover a patient 134 who is suffering from an acute injury or a medical condition that poses an imminent danger to the patient's health or life. The responders 150 activate the mobile devices 132 such that the mobile devices 132 commence capturing media data while the responders 150 assist the patient 134.

In various examples, the mobile devices 132 may be activated in a variety of ways. For example, the mobile devices 132 may be activated by pressing buttons (e.g., physical buttons or virtual buttons rendered on touch screens), issuing voice commands that the mobile devices 132 are configured to capture (e.g., via microphones) and recognize, or by performing gestures that input/output (I/O) devices associated with the mobile devices 132 are configured to capture and recognize (e.g., such as swiping a finger across a touch screen associated with one of the mobile devices 132, performing a hand gesture in view of a camera associated with one of the mobile devices 132, or agitating one of the mobile devices 132 in a manner that will be detected by an accelerometer associated therewith).

The media data captured by the mobile devices 132 may comprise, for example, audio data captured by microphones associated with the mobile devices 132, video data captured by digital cameras associated with the mobile devices 132, and other types of data captured by I/O) devices associated with the mobile devices 132 (e.g., a fingerprint image captured by a fingerprint scanner, a retina image captured by a retinal scanner, or other types of images captured by biometric devices). The mobile devices 132 may comprise, for example, smart phones, two-way radios (e.g., walkie talkies), body-worn cameras (BWCs), dashboard cameras, tablet computers, laptop computers, or other types of electronic devices that are capable of receiving user inputs (e.g., text, audio, or video) through I/O devices (e.g., microphones, touch screens, keyboards, computer mice, virtual reality headsets, biometric scanners, etc.) and storing those inputs into a digital format.

While the responders 150 assist the patient 134, the responders 150 may communicate verbally with each other and with the patient 134 to ascertain the patient 134's symptoms, diagnose the cause of the patient 134's current medical distress, collect data about the patient 134's medical history, and enumerate the types of treatment being provided to the patient 134 (e.g., such as medications being administered to the patient). The responders 150, being aware that the verbal communications are being recorded for the purpose of documenting medical data and for collecting samples of the patient 134's voice, may deliberately narrate what is being done as the patient 134 is assisted and deliberately ask the patient 134 numerous questions (e.g., to elicit responses from the patient 134 that both provide medical data about the patient and serve as speech samples of the patient 134's voice). The verbal communications between the responders 150 and the patient 134 are captured via microphones associated with the mobile devices 132 and, in one example, transmitted as a stream of audio data (which is one type of media data that is captured) to the servers 120 via the communications networks 102. In addition, a call identifier for the emergency call or location data from the mobile devices 132 (e.g., GPS coordinates) may be transmitted to the servers 120 to ensure that the audio data is associated with the emergency call for which the dispatcher 114 dispatched the responders 150 and with the scene 130.

Persons of skill in the art will also recognize that the audio data may be stored or transmitted in a variety of audio formats (or formats that combine video and audio). Some example formats may be, for example, Advanced Audio Coding (AAC), Moving Picture Experts Group (MPEG), MPEG Audio Layer III (MP3), Waveform Audio Format (WAV), Audio Interchange File Format (AIFF), Windows Media Audio (WMA), Audio/Video Interleaved (AVI), Pulse Code Modulation (PCM), Bitstream (RAW), or some other type of format. An exhaustive list of possible formats for audio data is beyond the scope of this disclosure.

When the audio data is received at the servers 120, a number of processes can be performed. For example, the speech-transcription software module 124 may generate a transcription of words that were spoken aloud and captured in the audio data (or a portion thereof). In addition, the speaker-diarization software module 125 may detect the number of people whose voices are captured in the audio data and generate metadata that indicates when each person detected was speaking in the audio data. For example, the speaker-diarization software module 125 may assign an identifier to each detected person and associate pairs of timestamps with each identifier. Each pair may include a first timestamp that indicates when the person represented by the identifier began speaking and a second timestamp that indicates when the person stopped speaking. Since a person may start speaking and stop speaking multiple times during a conversation, there may be many pairs of timestamps associated with each identifier (and therefore with the person identified thereby). Furthermore, the speaker-diarization software module 125 may infer roles of each detected person based on both acoustic cues (e.g., patient voices are more likely to sound agitated or strained, while responder voices are more likely to be calm) or linguistic cues. The speaker-diarization software module 125 may associate an inferred role (e.g., responder, patient, or bystander) with each identifier (and therefore with the person identified thereby) in the metadata.

There are many software tools available that may serve as the speech-transcription software module 124. For example, the Microsoft® Cognitive Services Speech Software Development Kit (SDK), Carnegie Mellon University (CMU) Sphinx, the Google® Cloud Speech Application Programming Interface (API), International Business Machines (IBM) Watson™ Speech to Text, Wit.ai, and the Houndify Application Programming Interface (API) are other examples of software tools that can be used to perform voice transcription. An exhaustive discussion of the software tools available for voice transcription is beyond the scope of this disclosure.

In addition, there are many software tools available that may perform the functions attributed to the speaker-diarization software module 125. For example, LIUM SpkDiarization DiarTk, ACLEW Diarization Virtual Machine (DiViMe), ALIZE-LIA_RAL, PyAnnote.audio, and Kaldi are a few software tools that can be used to perform speaker diarization (i.e., the process of mapping segments of audio to speakers). Joint Automatic Speech Recognition+Speaker Diarization (Joint ASR+SD) is a tool that can infer the roles of different people whose voices are detected. An exhaustive discussion of the software tools available for speaker diarization is beyond the scope of this disclosure.

Once the transcription has been generated, the natural-language-processing (NLP) software module 126 to the transcription to achieve several tasks. For example, the NLP software module 126 may identify a portion of the transcription that is associated with the patient 134. If there are no patients at the scene 130 other than the patient 134, then the portion might include the full transcription. If there are multiple patients at the scene 130, then the NLP software module 126 may identify the portion that is associated with the patient 134 (as opposed to some other patient) based on various features extracted from the transcript, such as when the patient 134's name was spoken (e.g., by the responders 150 or by the patient 134), which segments of the transcript are mapped to the patient 134's voice (e.g., as determined by the speaker-diarization software module 125), and which segments of the transcription appear to be responses to questions asked by the patient 134 or questions asked by the responders 150 that elicited responses from the patient. The NLP software module 126 may include such segments in the portion (note that the portion may comprise non-contiguous sections of the transcription if the responders 150 alternate between treating multiple patients at the scene 130). Also, the responders 150 may, intentionally and explicitly (e.g., because they know that the systems shown in FIG. 1 are being used), state when they are turning their attention from one patient to another as a matter of protocol. The NLP software module 126 may further perform part-of-speech (POS) tagging on the transcription to identify sentences in which the patient 134 is the subject of a verb, a referent of a pronoun, the direct object of a verb, or the indirect object of a verb. The NLP software module 126 may include such sentences in the portion.

Next, after identifying the portion of the transcription that is associated with the patient 134, the NLP software module 126 proceeds to extract medical data that pertains to the patient from the portion. The medical data may comprise, for example, symptoms experienced by the patient 134 (e.g., nausea, vomiting, bleeding, chest pain, abdominal pain, shortness of breath, dizziness, fever, hives, rash, swelling, headache, numbness, hallucinations, fatigue, etc.), types of injuries from which the patient 134 suffers (e.g., fracture, dislocated joint, puncture, abrasion, incision, laceration, burn, concussion, amputation, etc.), medical conditions that apply to the patient 134 (e.g., epilepsy, diabetes, pregnancy, asthma, osteogenesis imperfecta, eczema, autoimmune disorders, hemophilia, hypertension, allergies, etc.), foreign objects that are in the patient 134's body (e.g., pacemakers, replacement hip joints, replacement knee joints, stents, insulin pumps, dental implants, organ implants, cosmetic implants, orthopedic plates, shrapnel, etc.) and health metrics or characteristics of the patient (e.g., systolic blood pressure, diastolic blood pressure, heart rate, height, weight, blood type, age, prosthetics, etc.). Furthermore, the medical data may comprise substances taken by the patient 134 (e.g., alcohol, narcotics, opioids, tranquilizers, barbiturates, PCP, methamphetamine, marijuana, etc.), medications taken by the patient 134 (e.g., phosphodiesterase inhibitors, analgesics, antidepressants, antipsychotics, diuretics, stimulants, immunosuppressants, etc.), medications administered to the patient 134 by the responders 150 (e.g., epinephrine, albuterol, Naloxone, TXA, Warfarin, etc.). For substances and medications, the NLP software module 126 can further associate amounts (e.g., dosages) administered to the patient 134 by the responders 150 or taken by the patient 134 and timestamps that identify when those amounts were taken or administered with the names of each substance or medication.

The NLP software module 126 may further identify hazardous conditions that exist at the scene 130 and are mentioned in the transcription. Such descriptions may refer to conditions such as downed power lines, chemical spills, toxic clouds, icy roads, fires, explosive chemicals near potential ignition sources, active shooters, unstable structures (e.g., partially collapsed buildings or bridges), dangerous animals (e.g., rabid animals, poisonous snakes, and large wild predators), flooded streets, sharp debris, and other hazardous conditions.

There are many natural-language software tools that can be configured to perform the functions ascribed to the NLP software module 126 without undue experimentation. The Natural Language Toolkit (NLTK), SpaCy, TExtBlob, Textacy, and PyTorch-NLP are several examples of open-source tools that are available in the Python programming language, although there are many other NLP software tools available in many other programming languages. Such NLP tools may use many different techniques to extract features from natural-language data. For example, vectorization techniques such as Bag-of-Words, Term Frequency-Inverse Document Frequency (TF-IDF), Word2Vec, Global Vectors for word representation (GloVe), and FastText may be used to extract features. Techniques such as tokenization (e.g., N-gram tokenization), lemmatization, stemming, and part-of-speech tagging may be used to extract features in addition to, or as part of, vectorization techniques. Persons of skill in the art will understand that features may be digitally represented in a variety of ways. For example, a feature may be represented by an integer, a real number (e.g., decimal), an alphanumeric character, or a sequence of alphanumeric characters. Features may also be discretized, normalized (e.g., converted to a scale from zero to one), or preprocessed in other ways.

Features that are extracted from natural-language data can be used as input for machine-learning components of NLP tools that perform Named Entity Recognition (NER) to identify entities such as people, locations, companies, and dates that are mentioned in natural-language data. In addition, NLP tools can apply text summarization techniques (e.g., LexRank and TextRank) to those features to summarize content. Furthermore, NLP tools can apply techniques such as Latent Semantic Analysis (LSA), Probabilistic Latent Semantic Analysis (PLSA), Latent Dirichlet Allocation (LDA), and Correlated Topic Model (CTM) to the features to perform topic modeling. Persons of skill in the art will recognize that an in-depth discussion of the many NLP tools that are available and how those tools process input to generate output is beyond the scope of this disclosure.

As explained above, the speaker-diarization software module 125 outputs metadata that can be used to identify a subset of the audio data that includes speech uttered by the patient 134. The voice-vectorization software module 127 can be applied to that subset of the audio data to generate a voiceprint that represents the voice of the patient 134. The voiceprint may comprise a vector of feature values (e.g., actual parameters that map to formal parameters defined in a voiceprint template) derived from the subset of the audio data. There are many software tools that may be configured to perform the functions ascribed to the voice-vectorization software module 127. For example, OpenSpeaker, Kaldi, and ALIZE provide software tools that can generate and match voiceprints. An exhaustive list of software tools that can be configured to perform the functions ascribed to the voice-vectorization software module 127 is beyond the scope of this disclosure.

In addition to audio data, the media data may include image data (e.g., digital photos or digital video) that includes an image of the face of the patient 134. The facial-recognition software module 128 may detect and extract the image of the patient 134's face and associate the image with an identifier of the patient 134 (e.g., a name of the patient 134 extracted from the transcript by the NLP software module 126, an identifier assigned to the patient 134 by the speaker-diarization software module 125, or both). The facial-recognition software module 128 may apply video analytics to map the image of the patient's face to the identifier in a number of ways. For example, if the image data includes video, the facial-recognition software module 128 may use the metadata produced by the speaker-diarization software module 125 to identify a time window in the video in which the patient 134 is speaking and tag the face of the person whose mouth is moving in the video during that time window as the face of the patient 134. In addition, the facial-recognition software module 128 may infer who the patient 134 is based on the patient 134's clothing (e.g., the patient 134 may be wearing clothing that differs from uniforms worn by the responders 150).

There are many software tools that may be configured to perform functions ascribed to the facial-recognition software module 128. For example, Deepface, CompreFace, InsightFace, and FaceNet provide software tools that can recognize faces in image data. Functions such as associating clothing attributes with faces detected in video can be performed by tools such as that which is described in U.S. Pat. No. 9,195,883 (“Object Tracking and Best Shot Detection System”), which is hereby incorporated by reference. An exhaustive list of software tools that can be configured to perform the functions ascribed to the facial-recognition software module 128 is beyond the scope of this disclosure.

The voiceprint, the image of the patient 134's face, or a combination thereof may serve as a digital biometric identifier for the patient 134. If the responders 150 also have access to biometric scanners such as a fingerprint scanner or a retinal scanner, the responders 150 may also capture a fingerprint image or a retina image that may, individually or in combination with the voiceprint and the image of the patient 134's face, serve as the digital biometric identifier for the patient 134.

The digital biometric identifier for the patient 134 and the medical data for the patient 134 may be associated together in a digital data structure such as a Javascript Object Notation (JSON) object, a hash, or a tuple (e.g., for a database) by the aggregation-transmission software module 129. Next, the aggregation-transmission software module 129 signals networking hardware associated with the servers 120 (e.g., a network interface controller (NIC)) to transmit the digital data structure, the digital biometric identifier, and the medical data via the communications networks 102 to the computing device 172 (e.g., a smart phone, a tablet computer, a laptop computer, or a desktop computer) that is located at the treatment facility 170. The aggregation-transmission software module 129 may also include the call identifier for the emergency call or the location data from the mobile devices 132 with the digital biometric identifier, the media data, and the digital data structure. In addition, aggregation-transmission software module 129 may include the audio data so that the audio data will be available to be played via an electronic speaker in communication with the computing device 172 and the portion of the transcription from which the medical data was extracted.

If the NLP software module 126 identified any hazardous conditions that were mentioned in the transcription, the aggregation-transmission software module 129 signals the networking hardware associated with the servers 120 to transmit an alert to the a call-taking terminal 112 the communications networks 102 to notify the dispatcher 114 of the identified hazardous conditions. The alert may include the call identifier for the emergency call or the location data from the mobile devices 132 so that the dispatcher 114 will know that the alert maps to the scene 130 and emergency call to which the dispatcher 114 dispatched the responders 150. The dispatcher 114 may then inform additional responders 160 (who may be in route to the scene 130, but have not yet arrived) of the identified hazardous conditions.

When the ambulance 140 delivers the patient 134 to the treatment facility 170, staff members (e.g., nurses and doctors) at the treatment facility may receive the patient 134 and capture additional media data associated with the patient 134 using I/O devices that are in communication with the computing device 172. For example, the staff members may use a digital camera to capture a digital image of the patient 134's face, a microphone to capture speech uttered by the patient (if the patient 134 is able to speak), a fingerprint scanner to capture a fingerprint image from one or more of the patient 134's fingers, or a retinal scanner to capture a retina image of one or more of the patient 134's eyes. An additional digital biometric identifier may be extracted from the additional media data. For example, the additional media data may be uploaded to the servers 120 via the communications networks 102. The additional digital biometric identifier may be extracted by software at the servers 120 (e.g., by the voice-vectorization software module 127 or the facial-recognition software module 128 as described above) and compared (e.g., by the voice-vectorization software module 127 or the facial-recognition software module 128 at the servers 120) to determine whether the additional digital biometric identifier matches the digital biometric identifier that was extracted from the media data collected by the mobile devices 132 (e.g., at the scene 130 or in the ambulance 140). If the additional digital biometric identifier matches the digital biometric identifier, an electronic notification that indicates the match and comprises the medical data for the patient 134 is be sent to a staff member at the treatment facility 170 (e.g., via the computing device 172). In addition, the computing device 172 may generate a digital medical chart for the patient 134 and add one or more entries in the digital medical chart to reflect the medical data. For example, if a particular medical condition that affects the patient 134 is included in the medical data, the computing device 172 may add an entry comprising the name of the medical condition to the digital medical chart. Similarly, if the medical data specifies a medication that was administered to the patient 134 and a timestamp that identifies when the medication was administered to the patient 134, the computing device 172 may add an entry comprising the name of the medication and the timestamp to the digital medical chart.

While the examples described with respect to FIG. 1 depict certain software components as being executed via processors, memory, and other hardware at the servers 120, persons of skill in the art will understand that various software components that are described as executing on the servers 120 may be executed on the mobile devices 132 or the computing device 172 without departing from the spirit and scope of this disclosure. For example, the speech-transcription software module 124, the speaker-diarization software module 125, the natural-language-processing (NLP) software module 126, the voice-vectorization software module 127, the facial-recognition software module 128, the aggregation-transmission software module 129, or components thereof may reside partially or wholly on the mobile devices 132 or the computing device 172 without departing from the spirit and scope of this disclosure.

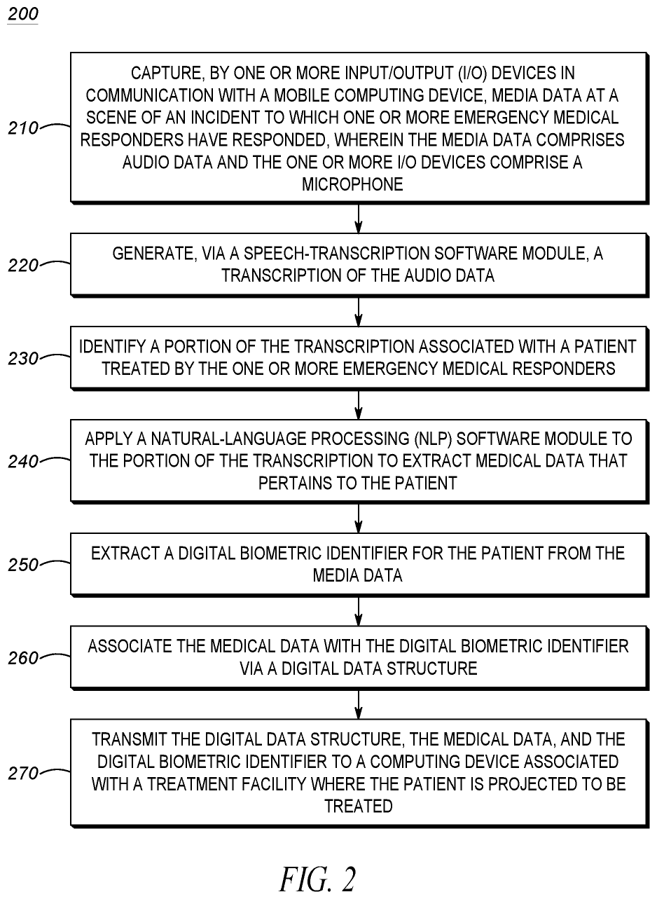

FIG. 2 illustrates functionality 200 for systems disclosed herein, according to one example. The functionality 200 does not have to be performed in the exact sequence shown. Also, various blocks may be performed in parallel rather than in sequence. Accordingly, the elements of the functionality 200 are referred to herein as “blocks” rather than “steps.” The functionality 200 can be executed as instructions on a machine (e.g., by one or more processors), where the instructions are stored on a transitory or non-transitory computer-readable storage medium. While only seven blocks are shown in the functionality 200, the functionality 200 may comprise other actions described herein. Also, in some examples, some of the blocks shown in the functionality 200 may be omitted without departing from the spirit and scope of this disclosure.

As shown in block 210, the functionality 200 includes capturing, by one or more input/output (I/O) devices in communication with a mobile computing device, media data at a scene of an incident to which one or more emergency medical responders have responded, wherein the media data comprises audio data and the one or more I/O devices comprise a microphone.

As shown in block 220, the functionality 200 includes generating, via a speech-transcription software module, a transcription of the audio data.

As shown in block 230, the functionality 200 includes identifying a portion of the transcription associated with a patient treated by the one or more emergency medical responders.

As shown in block 240, the functionality 200 includes applying a natural-language processing (NLP) software module to the portion of the transcription to extract medical data that pertains to the patient. The medical data may comprise, for example, a name of a medication, an amount of the medication administered to the patient, and a timestamp identifying when the medication was administered to the patient. In addition, the medical data may comprise a name of a medical condition that affects the patient.

As shown in block 250, the functionality 200 includes extracting a digital biometric identifier for the patient from the media data. Extracting the digital biometric identifier of the patient may comprise, for example, applying a speaker-diarization software module to the audio data to identify a subset of the audio data that includes speech uttered by the patient; and generating, via a voice-vectorization software module, a voiceprint that represents a voice of the patient as captured in the subset of the audio data, wherein the digital biometric identifier comprises the voiceprint. Also, the one or more one or more I/O may comprise a digital camera, the media data may comprise image data, and extracting the digital biometric identifier of the patient may comprise applying a facial-recognition software module to the image data to extract an image of a face of the patient, wherein the digital biometric identifier comprises the image of the face of the patient.

As shown in block 260, the functionality 200 includes associating the medical data with the digital biometric identifier via a digital data structure.

As shown in block 270, the functionality 200 includes transmitting the digital data structure, the medical data, and the digital biometric identifier to a computing device associated with a treatment facility where the patient is projected to be treated. In addition, the functionality 200 may include transmitting the audio data and the portion of the transcription to the computing device associated with the treatment facility along with the digital data structure, the medical data, and the digital biometric identifier.

The functionality 200 may also include further applying the NLP software module to the transcription to identify a description of a hazardous condition at the scene that poses a physical danger to the one or more emergency medical responders; and transmitting an alert to an additional computing device associated with a dispatcher to notify the dispatcher of the hazardous condition and instruct the dispatcher to advise at least one additional responder of the hazardous condition.

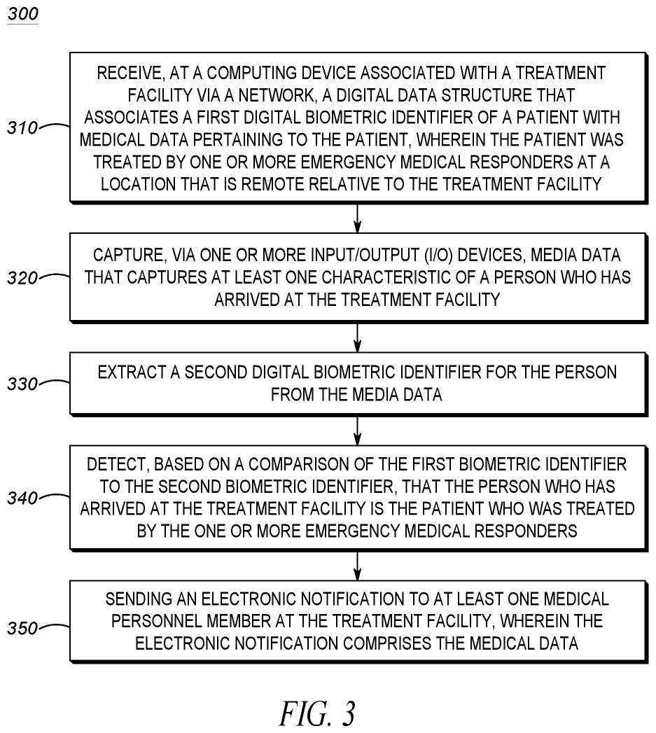

FIG. 3 illustrates additional functionality 300 for systems disclosed herein, according to one example. The functionality 300 does not have to be performed in the exact sequence shown. Also, various blocks may be performed in parallel rather than in sequence. Accordingly, the elements of the functionality 300 are referred to herein as “blocks” rather than “steps.” The functionality 300 can be executed as instructions on a machine (e.g., by one or more processors), where the instructions are stored on a transitory or non-transitory computer-readable storage medium. While only five blocks are shown in the functionality 300, the functionality 300 may comprise other actions described herein. Also, in some examples, some of the blocks shown in the functionality 300 may be omitted without departing from the spirit and scope of this disclosure.

As shown in block 310, the functionality 300 includes receiving, at a computing device associated with a treatment facility via a network, a digital data structure that associates a first digital biometric identifier of a patient with medical data pertaining to the patient, wherein the patient was treated by one or more emergency medical responders at a location that is remote relative to the treatment facility. The first digital biometric identifier may comprise, for example, a voiceprint that represents a voice of the patient or an image of the patient.

As shown in block 320, the functionality 300 includes capturing, via one or more input/output (I/O) devices, media data that captures at least one characteristic of a person who has arrived at the treatment facility. The one or more I/O devices may comprise, for example, a microphone. Capturing the media data may comprise capturing speech uttered by the person who has arrived at the treatment facility via the microphone. The one or more I/O devices may also comprise a digital camera and capturing the media data may comprise capturing an image of a face of the person who has arrived at the treatment facility via the digital camera.

As shown in block 330, the functionality 300 includes extracting a second digital biometric identifier for the person from the media data. Extracting the second digital identifier may comprise generating, via a voice-vectorization software module, a voiceprint that represents a voice of the person as captured in the media data.

As shown in block 340, the functionality 300 includes detecting, based on a comparison of the first biometric identifier to the second biometric identifier, that the person who has arrived at the treatment facility is the patient who was treated by the one or more emergency medical responders. The comparison of the first digital biometric identifier to the second digital biometric identifier may be performed by a facial-recognition software module, a voice-vectorization software module, or a combination thereof.

As shown in block 350, the functionality 300 includes sending an electronic notification to at least one medical personnel member at the treatment facility, wherein the electronic notification comprises the medical data.

The medical data may comprise, for example, a name of a medication, an amount of the medication administered to the patient, and a timestamp identifying when the medication was administered to the patient. In addition, the medical data may comprise a name of a medical condition that affects the patient.

The functionality 300 may further include adding an entry comprising the name of the medication and the timestamp to a digital medical chart for the person at the treatment facility and adding an entry comprising the name of the medical condition to the digital medical chart.

The functionality 300 may further include receiving, at the computing device associated with the treatment facility, audio data from which the medical data was extracted; and playing at least a portion of the audio data from a speaker in communication with the computing device.

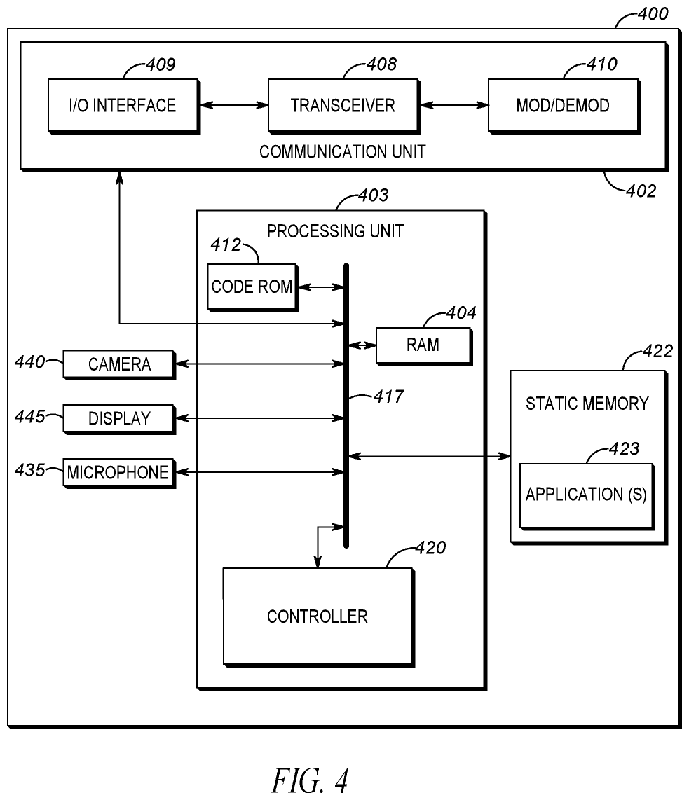

FIG. 4 illustrates a schematic block diagram of a computing device 400, according to one example. The computing device 400 may be configured to capture media data that includes audio data, generate a transcription of the audio data, extract medical data for a patient from the transcription, extract a digital biometric identifier for the patient, generate a data structure that associates the digital biometric identifier with the medical data, and transmit the data structure, the digital biometric identifier, and the medical data (e.g., as described above with respect to FIGS. 1-3) via one or more different types of networks (e.g., networks with which the transceivers 408 may be adapted to communicate, as discussed in further detail below).

The computing device 400 may comprise a cellular phone (e.g., a smart phone), a satellite phone, a Voice over Internet Protocol (VoIP) phone, a two-way radio, or a computer (e.g., a workstation, a laptop, a mobile tablet computer or a desktop computer) that is equipped with peripherals for recording a digital witness statement (e.g., a microphone 435, and a camera 440).

As depicted, the computing device 400 comprises a communication unit 402, a processing unit 403 (e.g., a processor), a Random-Access Memory (RAM) 404, one or more transceivers 408 (which may be wireless transceivers), one or more wired or wireless input/output (I/O) interfaces 409, a combined modulator/demodulator 410 (which may comprise a baseband processor), a code Read-Only Memory (ROM) 412, a common data and address bus 417, a controller 420, and a static memory 422 storing one or more applications 423.

The computing device 400 may also include a camera 440, a display 445, and a microphone 435 such that a user may use the computing device 400 to capture audio data (e.g., speech uttered by a patient or by responders) and image data (e.g., a digital image of a patient).

As shown in FIG. 4, the computing device 400 includes the communication unit 402 communicatively coupled to the common data and address bus 417 of the processing unit 403. The processing unit 403 may include the code Read Only Memory (ROM) 412 coupled to the common data and address bus 417 for storing data for initializing system components. The processing unit 403 may further include the controller 420 coupled, by the common data and address bus 417, to the Random-Access Memory 404 and the static memory 422. Persons of skill in the art will recognize that other configurations (e.g., configurations that include multiple buses) may also be used without departing from the spirit and scope of this disclosure.

The communication unit 402 may include one or more wired or wireless input/output (I/O) interfaces 409 that are configurable to communicate with other components and devices. For example, the communication unit 402 may include one or more transceivers 408 or wireless transceivers may be adapted for communication with one or more communication links or communication networks used to communicate with other components or computing devices. For example, the one or more transceivers 408 may be adapted for communication with one or more of the Internet (including public and private Internet Protocol (IP) networks), a private IP wide area network (WAN) including a National Emergency Number Association (NENA) i3 Emergency Services Internet Protocol (IP) network (ESInet), a Bluetooth network, a Wi-Fi network, for example operating in accordance with an Institute of Electrical and Electronics Engineers (IEEE) 802.11 standard (e.g., 802.11a, 802.11b, 802.11g, 802.11ax), a 3G standard network (including Global System for Mobiles (GSM) and Code Division Multiple Access (CDMA) standards), an LTE (Long-Term Evolution) network or other types of GSM networks, a 5G (including a network architecture compliant with, for example, the Third Generation Partnership Project (3GPP) Technical Specification (TS) 23 specification series and a new radio (NR) air interface compliant with the 3GPP TS 38 specification series) standard network, a Citizens Broadband Radio Service (CBRS), Worldwide Interoperability for Microwave Access (WiMAX) network, for example operating in accordance with an IEEE 802.16 standard, a landline telephonic network, a Low Earth Orbit (LEO) network (e.g., for satellite phones or Internet connection), a Geosynchronous Orbit (GEO) network (e.g., for satellite phones), an Evolution-Data Optimized (EV-DO) network, an Enhanced Data Rates for Global Evolution (EDGE) network, or another similar type of wireless network. Hence, the one or more transceivers 408 may include, but are not limited to, a cell phone transceiver, a Bluetooth transceiver, a CBRS transceiver, a Wi-Fi transceiver, a WiMAX transceiver, or another similar type of wireless transceiver configurable to communicate via a wireless radio network.

The one or more transceivers 408 may also comprise one or more wired transceivers, such as an Ethernet transceiver, a USB (Universal Serial Bus) transceiver, or similar transceiver configurable to communicate via a twisted pair wire, a coaxial cable, a fiber-optic link, or a similar physical connection to a wired network. The one or more transceivers 408 are also coupled to a combined modulator/demodulator 410.

The controller 420 may include ports (e.g., hardware ports) for coupling to other hardware components or systems (e.g., components and systems described with respect to FIG. 1). The controller 420 may also comprise one or more logic circuits, one or more processors, one or more microprocessors, one or more application-specific integrated circuits (ASICs), one or more field-programmable gate arrays (FPGAs), or other electronic devices.

The static memory 422 is a non-transitory machine readable medium that stores machine readable instructions to implement one or more programs or applications (e.g., such as the speech-transcription software module 124, the speaker-diarization software module 125, the NLP software module 126, the voice-vectorization software module 127, the facial-recognition software module 128, and the aggregation-transmission software module 129 described with respect to FIG. 1). Example machine readable media include a non-volatile storage unit (e.g., Erasable Electronic Programmable Read Only Memory (EEPROM), Flash Memory, etc.), or a volatile storage unit (e.g. random-access memory (RAM)). In the example of FIG. 4, programming instructions (e.g., machine readable instructions) that implement the functional teachings of the computing device 400 as described herein are maintained, persistently, at the static memory 422 and used by the controller 420, which makes appropriate utilization of volatile storage during the execution of such programming instructions.

When the controller 420 executes the one or more applications 423, the controller 420 is enabled to perform one or more of the aspects of the present disclosure set forth earlier in the present specification (e.g., the computing device blocks set forth in FIG. 3). The one or more applications 423 may include programmatic algorithms, and the like, that are operable to perform electronic functions described with respect to FIGS. 1-3.

Examples are herein described with reference to flowchart illustrations and/or block diagrams of methods, apparatus (systems) and computer program products according to various examples. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a special purpose and unique machine, such that the instructions, which execute via the processor of the computer or other programmable data processing apparatus, create means for implementing the functions/acts specified in the flowchart and/or block diagram block or blocks. The methods and processes set forth herein need not, in some examples, be performed in the exact sequence as shown and likewise various blocks may be performed in parallel rather than in sequence. Accordingly, the elements of methods and processes are referred to herein as “blocks” rather than “steps.”

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the flowchart and/or block diagram block or blocks.

The computer program instructions may also be loaded onto a computer or other programmable data processing apparatus that may be on or off-premises, or may be accessed via the cloud in any of a software as a service (SaaS), platform as a service (PaaS), or infrastructure as a service (IaaS) architecture so as to cause a series of operational blocks to be performed on the computer or other programmable apparatus to produce a computer implemented process such that the instructions which execute on the computer or other programmable apparatus provide blocks for implementing the functions/acts specified in the flowchart and/or block diagram block or blocks. It is contemplated that any part of any aspect or example discussed in this specification can be implemented or combined with any part of any other aspect or example discussed in this specification.

As should be apparent from this detailed description above, the operations and functions of the electronic computing device are sufficiently complex as to require their implementation on a computer system, and cannot be performed, as a practical matter, in the human mind. Electronic computing devices such as set forth herein are understood as requiring and providing speed and accuracy and complexity management that are not obtainable by human mental steps, in addition to the inherently digital nature of such operations (e.g., a human mind cannot interface directly with RAM or other digital storage, cannot transmit or receive electronic messages, electronically encoded video, electronically encoded audio, etc., and cannot generate voiceprints, among other features and functions set forth herein).

In the foregoing specification, specific examples have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a,” “has . . . a,” “includes . . . a,” or “contains . . . a” does not, without more constraints, preclude the existence of additional identical elements in the process, method, article, or apparatus that comprises, has, includes, or contains the element. The terms “a” and “an” are defined as one or more unless explicitly stated otherwise herein. The terms “substantially,” “essentially,” “approximately,” “about,” or any other version thereof, are defined as being close to as understood by one of ordinary skill in the art, and in one non-limiting example the term is defined to be within 10%, in another example within 5%, in another example within 1%, and in another example within 0.5%. The term “one of,” without a more limiting modifier such as “only one of,” and when applied herein to two or more subsequently defined options such as “one of A and B” should be construed to mean an existence of any one of the options in the list alone (e.g., A alone or B alone) or any combination of two or more of the options in the list (e.g., A and B together).

A device or structure that is “configured” in a certain way is configured in at least that way, but may also be configured in ways that are not listed.

The terms “coupled,” “coupling,” or “connected” as used herein can have several different meanings depending on the context in which these terms are used. For example, the terms coupled, coupling, or connected can have a mechanical or electrical connotation. For example, as used herein, the terms coupled, coupling, or connected can indicate that two elements or devices are directly connected to one another or connected to one another through intermediate elements or devices via an electrical element, electrical signal or a mechanical element depending on the particular context.

It will be appreciated that some examples may comprise one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an example can be implemented as a computer-readable storage medium having computer readable code stored thereon for programming a computer (e.g., comprising a processor) to perform a method as described and claimed herein. Any suitable computer-usable or computer readable medium may be utilized. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. In the context of this document, a computer-usable or computer-readable medium may be any medium that can contain, store, communicate, propagate, or transport the program for use by or in connection with the instruction execution system, apparatus, or device.

Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation. For example, computer program code for carrying out operations of various examples may be written in an object oriented programming language such as Java, Smalltalk, C++, Python, or the like. However, the computer program code for carrying out operations of various examples may also be written in conventional procedural programming languages, such as the “C” programming language or similar programming languages. The program code may execute entirely on a computer, partly on the computer, as a stand-alone software package, partly on the computer and partly on a remote computer or server or entirely on the remote computer or server. In the latter scenario, the remote computer or server may be connected to the computer through a local area network (LAN) or a wide area network (WAN), or the connection may be made to an external computer (for example, through the Internet using an Internet Service Provider).

The Abstract of the Disclosure is provided to allow the reader to ascertain the nature of the technical disclosure quickly. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus the following claims are hereby incorporated into the Detailed Description, with each claim standing on its own as a separately claimed subject matter.

What is claimed is:

1. A method comprising:
capturing, by one or more input/output (I/O) devices in communication with a mobile computing device, media data at a scene of an incident to which one or more emergency medical responders have responded, wherein the media data comprises audio data and the one or more I/O devices comprise a microphone;
generating, via a speech-transcription software module, a transcription of the audio data;
identifying a portion of the transcription associated with a patient treated by the one or more emergency medical responders;
applying a natural-language processing (NLP) software module to the portion of the transcription to extract medical data that pertains to the patient;
extracting a digital biometric identifier for the patient from the media data;
associating the medical data with the digital biometric identifier via a digital data structure; and
transmitting the digital data structure, the medical data, and the digital biometric identifier to a computing device associated with a treatment facility where the patient is projected to be treated.

2. The method of claim 1, wherein extracting the digital biometric identifier of the patient comprises:
applying a speaker-diarization software module to the audio data to identify a subset of the audio data that includes speech uttered by the patient; and
generating, via a voice-vectorization software module, a voiceprint that represents a voice of the patient as captured in the subset of the audio data, wherein the digital biometric identifier comprises the voiceprint.

3. The method of claim 1, wherein the one or more one or more I/O devices comprise a digital camera, the media data comprises image data, and extracting the digital biometric identifier of the patient comprises:
applying a facial-recognition software module to the image data to extract an image of a face of the patient, wherein the digital biometric identifier comprises the image of the face.

4. The method of claim 1, wherein the medical data comprises a name of a medication, an amount of the medication administered to the patient, and a timestamp identifying when the medication was administered to the patient.

5. The method of claim 1, wherein the medical data comprises a name of a medical condition that affects the patient.

6. The method of claim 1, further comprising:
transmitting the audio data and the portion of the transcription to the computing device associated with the treatment facility along with the digital data structure, the medical data, and the digital biometric identifier.

7. The method of claim 1, further comprising:
further applying the NLP software module to the transcription to identify a description of a hazardous condition at the scene that poses a physical danger to the one or more emergency medical responders; and
transmitting an alert to an additional computing device associated with a dispatcher to notify the dispatcher of the hazardous condition and instruct the dispatcher to advise at least one additional responder of the hazardous condition.

8. A method comprising:
receiving, at a computing device associated with a treatment facility via a network, a digital data structure that associates a first digital biometric identifier of a patient with medical data pertaining to the patient, wherein the patient was treated by one or more emergency medical responders at a location that is remote relative to the treatment facility;
capturing, via one or more input/output (I/O) devices, media data that captures at least one characteristic of a person who has arrived at the treatment facility;
extracting a second digital biometric identifier for the person from the media data;
detecting, based on a comparison of the first biometric identifier to the second biometric identifier, that the person who has arrived at the treatment facility is the patient who was treated by the one or more emergency medical responders; and
sending an electronic notification to at least one medical personnel member at the treatment facility, wherein the electronic notification comprises the medical data.

9. The method of claim 8, wherein:
the one or more I/O devices comprise a microphone;
capturing the media data comprises capturing speech uttered by the person who has arrived at the treatment facility via the microphone; and
extracting the second digital biometric identifier comprises generating, via a voice-vectorization software module, a voiceprint that represents a voice of the person as captured in the media data, wherein the first digital biometric identifier comprises a voiceprint that represents a voice of the patient.

10. The method of claim 8, wherein:
the one or more I/O devices comprise a digital camera;
capturing the media data comprises capturing an image of a face of the person who has arrived at the treatment facility via the digital camera;
the second digital biometric identifier comprises the image of the face of the person;
the first digital biometric identifier comprises an image of a face of the patient; and
the comparison of the first digital biometric identifier to the second digital biometric identifier is performed by a facial-recognition software module.

11. The method of claim 8, wherein the medical data comprises a name of a medication and a timestamp identifying when the medication was administered to the patient, and wherein the method further comprises:
adding an entry comprising the name of the medication and the timestamp to a digital medical chart for the person at the treatment facility.

12. The method of claim 8, wherein the medical data comprises a name of a medical condition that affects the patient, and wherein the method further comprises:
adding an entry comprising the name of the medical condition to a digital medical chart for the person at the treatment facility.

13. The method of claim 8, further comprising:
receiving, at the computing device associated with the treatment facility, audio data from which the medical data was extracted; and
playing at least a portion of the audio data from a speaker in communication with the computing device.

14. A non-transitory computer-readable storage medium containing instructions that, when executed by one or more processors, perform a set of actions comprising:
capturing, by one or more input/output (I/O) devices in communication with a mobile computing device, media data at a scene of an incident to which one or more emergency medical responders have responded, wherein the media data comprises audio data and the one or more I/O devices comprise a microphone;
generating, via a speech-transcription software module, a transcription of the audio data;
identifying a portion of the transcription associated with a patient treated by the one or more emergency medical responders;
applying a natural-language processing (NLP) software module to the portion of the transcription to extract medical data that pertains to the patient;
extracting a digital biometric identifier for the patient from the media data;
associating the medical data with the digital biometric identifier via a digital data structure; and
transmitting the digital data structure, the medical data, and the digital biometric identifier to a computing device associated with a treatment facility where the patient is projected to be treated.

15. The non-transitory computer-readable storage medium of claim 14, wherein extracting the digital biometric identifier of the patient comprises:
applying a speaker-diarization software module to the audio data to identify a subset of the audio data that includes speech uttered by the patient; and
generating, via a voice-vectorization software module, a voiceprint that represents a voice of the patient as captured in the subset of the audio data, wherein the digital biometric identifier comprises the voiceprint.

16. The non-transitory computer-readable storage medium of claim 14, wherein the one or more one or more I/O devices comprise a digital camera, the media data comprises image data, and extracting the digital biometric identifier of the patient comprises:
applying a facial-recognition software module to the image data to extract an image of a face of the patient, wherein the digital biometric identifier comprises the image of the face of the patient.

17. The non-transitory computer-readable storage medium of claim 14, wherein the medical data comprises a name of a medication, an amount of the medication administered to the patient, and a timestamp identifying when the medication was administered to the patient.

18. The non-transitory computer-readable storage medium of claim 14, wherein the medical data comprises a name of a medical condition that affects the patient.

19. The non-transitory computer-readable storage medium of claim 14, wherein the set of actions further comprises:
transmitting the audio data and the portion of the transcription to the computing device associated with the treatment facility along with the digital data structure, the medical data, and the digital biometric identifier.

20. The non-transitory computer-readable storage medium of claim 14, further comprising:
further applying the NLP software module to the transcription to identify a description of a hazardous condition at the scene that poses a physical danger to the one or more emergency medical responders; and
transmitting an alert to an additional computing device associated with a dispatcher to notify the dispatcher of the hazardous condition and instruct the dispatcher to advise at least one additional responder of the hazardous condition.
